(19) United States
(12) Patent Application Publication
(10) Pub. No.: US 2021/0151135 A1
(43) Pub. Date: May 20, 2021

(54) PROCESSING DATA RECORDS AND SEARCHING DATA STRUCTURES THAT ARE STORED IN HARDWARE MEMORY AND THAT ARE AT LEAST PARTLY GENERATED FROM THE PROCESSED DATA RECORDS IN GENERATING AN ADAPTIVE USER INTERFACE

(71) Applicant: Universal Research Solutions, LLC, Columbia, MO (US)

(72) Inventors: Ali Adel Hussam, Columbia, MO (US); Nathan Bleigh, Kansas City, MO (US)

(73) Assignee: Universal Research Solutions, LLC, Columbia, MO (US)

(21) Appl. No.: 16/640,007

(22) PCT Filed: Aug. 20, 2018
(86) PCT No.: PCT/US2018/000363

§ 371 Date: Feb. 18, 2020

Related U.S. Application Data
(60) Provisional application No. 62/547,733, filed on Aug. 18, 2017.

(51) Int. Cl. G16H 10/20; G16H 10/60; G06F 16/242; G06F 16/28; G06F 16/2457

(52) U.S. Cl. CPC G16H 10/20; G16H 10/60; G06F 16/24573; G06F 16/285; G06F 16/244

(57) ABSTRACT

A data processing system is disclosed that includes an input port that aggregates data extracted from multiple different data records that have been retrieved from multiple distributed data sources, a classification engine that classifies the aggregated data into a first data type and a second data type, an executable logic engine that defines a first logical mapping from fields in the first data type to respective regions of a graphical user interface and that further defines a second logical mapping from the fields in the second data type to respective regions of the graphical user interface, and an output port that receives a request to display the graphical user interface from a user device, and provides data for the graphical user interface that when rendered on a display device, renders visual representations of the respective regions populated with data based on a particular logical mapping selected by the output port.

### BACKGROUND

Medical forms are used to collect data and information regarding a patient's symptoms and conditions. One technique for preparing a medical form is to manually edit a pre-existing form (e.g., a form existing in Microsoft Word™ format) with new or customized questions. The form is then sent to review boards for review through a physical or electronic mailing. Additionally, once a form has been finalized, it may be presented to a patient, study participant or other individual (collectively referred to as “patients” herein, without limitation, for purposes of convenience). For example, physicians may present patients with the forms when the patient visits the physician's office. Additionally, hardcopy (i.e., paper) versions of medical forms may be distributed to patients for completion. For patients who have not completed medical forms prior to the patient's examination, the patient may often complete the medical form at the physician's office by filling out a hardcopy of the form.

Frequently, the patient's responses to the questions included in the medical forms are entered into a computerized system by medical personnel. In this case, in order for a physician to review the patient's responses, the physician may access the computerized system and view the answers to the questions, which is often a lengthy process of reviewing individual questions.

### BACKGROUND

According to one innovative aspect of the present disclosure, a data processing system for processing data records and searching data structures at least partly generated from the processed data records is disclosed. The data process system may include an input port that retrieves, from multiple, different data stores, data records, with each data record representing a medical study instrument, an extraction engine that processes the data records by extracting specified portions of data included in each of the data records retrieved over the input port, a central data storage repository that aggregates the extracted data that has been extracted from the data records representing respective multiple different medical study instruments, a classification engine that classifies a first portion of the aggregated data into a first type of data and a second portion of the aggregated data into a second type of data, an executable logic engine that defines a first logical mapping from each of a plurality of fields in the first type of data to one or more respective regions of a graphical user interface and that further defines a second logical mapping from each of a plurality of fields in the second type of data to one or more respective regions of the graphical user interface, and an output port that: receives a request to display the graphical user interface from a user of a user device, wherein the request includes a key identifier, searches data structures representing the first and second logical mapping, wherein at least one of the searched data records includes a key identifier, responsive to searching, selects either the first logical mapping or the second logical mapping based on the received key identifier, and provides data for the graphical user interface that when rendered on a display device renders one or more visual representations of the one or more respective regions populated with data based on the selected logical mapping.

Other versions include corresponding methods, apparatus, and computer programs to perform the actions of methods defined by instructions encoded on computer storage devices.

These and other versions may optionally include one or more of the following features. For instance, in some implementations, at least one first region of the graphical user interface is populated with one or more question-answer combinations based on the selected logical mapping.

In some implementations, at least one second region of the graphical user interface is populated with data that is indicative of the number of people who have provided the same answer to the same question set forth in the particular question-answer combination.

In some implementations, the second region of the graphical user interface may further include a first displayed value that is indicative of the number of people at a particular medical facility that provided the same answer to the same question set forth in the particular question-answer combination, a second displayed value that is indicative of the number of people at multiple different medical facilities that provided the same answer to the same questions set forth in the particular question-answer combination, and a third displayed value that is indicative of the difference between the first displayed value and the second displayed value.

In some implementations, the output port, or other component of the user interface generator, may configured to determine whether the third displayed value exceeds a predetermined threshold, and in response to a determination that the third displayed value exceeds a predetermined threshold, the output port can highlight a predetermined proximity of the graphical user interface associated with the third displayed value using a first color.

In some implementations, the output port, or other component of the user interface generator, may be configured to determine whether the third displayed value falls below a predetermined threshold, and in response to a determination that the third displayed value falls below a predetermined threshold, the output port can highlight a predetermined proximity of the graphical user interface associated with the third displayed value using a second color that is different than the first color.

According to one implementation, the subject matter of this specification may be embodied in a method for generating output based on multi-modal data that is aggregated from a plurality of disparate data sources using an adaptive user interface is disclosed. The method may include actions of generating a central data storage repository that aggregates data that has been extracted from multiple different medical study instruments, wherein the aggregated data is classified into a first type of data and a second type of data, defining a first logical mapping from each of a plurality of fields in the first type of data to one or more respective regions of a graphical user interface, defining a second logical mapping from each of a plurality of fields in the second type of data to one or more respective regions of the graphical user interface, receiving a request to display the graphical user interface from a user of a user device, wherein the request includes an entity identifier, selecting either the first logical mapping or the second logical mapping based on the received entity identifier, and providing the graphical user interface for display, wherein the one or more respective regions are populated with data based on the selected logical mapping.

Other versions include corresponding systems, apparatus, and computer programs to perform the actions of methods defined by instructions encoded on computer storage devices.

These and other versions may optionally include one or more of the following features. For instance, in some implementations, generating a central repository that aggregates data that has been extracted from multiple different medical study instruments may include obtaining a plurality of populated medical study instruments from a plurality of different computers, extracting one or more question-answer pairs from the obtained medical study instruments, determining a classification of each extracted question-answer pairs, and saving the question-answer pair into an aggregated data storage area based on the determined classification.

In some implementations, at least one first region of the graphical user interface is populated with one or more question-answer combinations based on the selected logical mapping.

In some implementations, at least one second region of the graphical user interface is populated with data that is indicative of the number of people who have provided the same answer to the same question set forth in the particular question-answer combination.

In some implementations, the second region of the graphical user interface may include a first displayed value that is indicative of the number of people at a particular medical facility that provided the same answer to the same question set forth in the particular question-answer combination, a second displayed value that is indicative of the number of people at multiple different medical facilities that provided the same answer to the same questions set forth in the particular question-answer combination, and a third displayed value that is indicative of the difference between the first displayed value and the second displayed value.

In some implementations, the method may also include determining whether the third displayed value exceeds a predetermined threshold, and in response to determining that the third displayed value exceeds a predetermined threshold, highlighting a predetermined proximity of the graphical user interface associated with the third displayed value using a first color.

In some implementations, determining whether the third displayed value falls below a predetermined threshold, and in response to determining that the third displayed value falls below a predetermined threshold, highlighting a predetermined proximity of the graphical user interface associated with the third displayed value using a second color that is different than the first color.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an example of a contextual diagram of an example of a system for implementing adaptive user interfaces.

FIG. 2 is a flowchart of an example of a process for implementing adaptive user interfaces.

FIG. 3 is an example of a scorecard that is generated for a medical facility.

FIG. 4 is an example of a scorecard that may be generated for a medical provider.

### WRITTEN DESCRIPTION

In some implementations, the subject matter of the present disclosure provides a system and method that can be used to provide views of aggregated data from multiple different sources using an adaptive user interface. The subject matter of the present disclosure improves the functionality of a machine by increasing the efficiency with which the data from multiple disparate sources can be accessed by an adaptive user interface. A user interface generator can access data structures representing predefined logical mappings between aggregated data stores and user interface regions in order to efficiently populate the adaptive user interface described herein. Hereinafter, “logical mappings” may refer to data structures representing predefined logical mappings for purposes of convenience and without limitation.

The tandem of the predefined logical mappings and the aggregated data store reduce the latency involved in obtaining data to populate an adaptive user interface using data that originated on multiple disparate source computers. The predefined logical mappings and the aggregated data stores also conserve network bandwidth as data to populate the adaptive user interface need only be obtained from a single aggregated data source using the predefined logical mappings, as opposed to sending data search requests, and receiving search results from, multiple disparate data sources to populate the adaptive user interface each time the data from such disparate data sources is required.

The adaptive user interface includes one or more regions that can be used to output the aggregated data that is stored in the central storage repository. The particular aggregated data that is used to populate each region may be determined by selecting one or more predefined logical mappings of aggregated data to interface region. Particular logical mappings may be selected based on the user that requests the populated user interface.

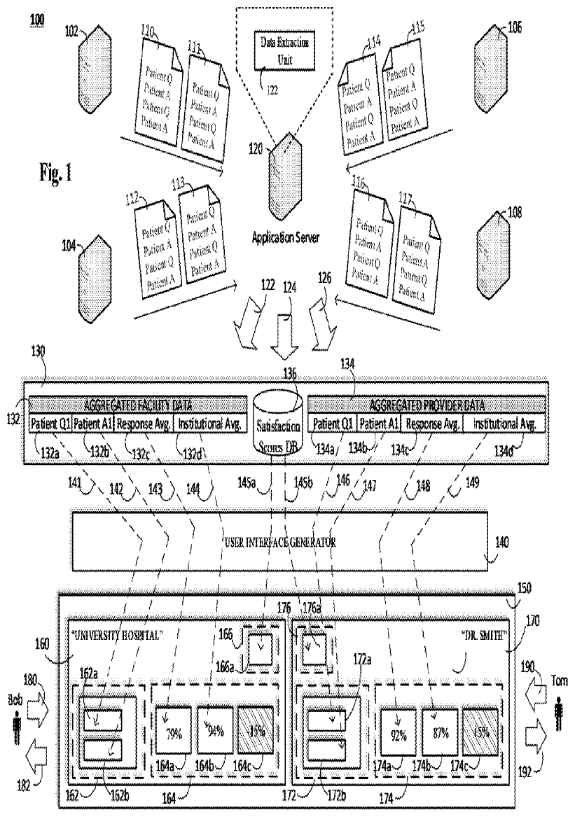

FIG. 1 is an example of a contextual diagram of an example of a system 100 for implementing adaptive user interfaces. The system 100 may include an application server 120, data extraction engine 122, a centralized data storage repository 130, a user interface generator 140, and an adaptive user interface 150.

The application server 120 may include one or more server computers that are configured to obtain a plurality of different data records, including, e.g., data records representing medical study instruments such as medical study instruments 110, 111, 112, 113, 114, 115, 116, 117 from a plurality of disparate computers 102, 104, 106, 108. For example, the application server 120 may include an input port that retrieves, from multiple different data stores such as disparate computers 102, 104, 106, 108, data records with each data records representing one or more portions of a medical study instrument. The input port of the application server 120 may include a network interface port, PCI port, communications bus, or a combination thereof, or any other interface and set of interconnections that facilitate receipt of data records from one or more different data stores such as disparate computers 102, 104, 106, 108. In some implementations, the entire medical study instrument may be represented by a single data record. Alternatively, a single medical study instrument may be represented by multiple data records. Hereinafter, a “medical study instrument” may refer to a data record representing a medical study instrument for purposes of convenience and without limitation.

The application server 120 may include a data extraction engine 122 that can identify and extract information from the obtained medical Study instruments. In one implementation, the data extraction engine 120 may include one or more software components, one or more hardware components, or a combination of both (e.g., one or more hardware processors executing programmed logic), that reside in one or more servers such as the application server 120 and can communicate with one or more other computers 102, 104, 106, 108 to obtain each respective medical study instrument of the plurality of medical study instruments. By way of example, the data extraction engine may process obtained data records including data that represents one or more medical study instruments by extracting specified portions of data included in each of the data records retrieved over the input port of the application server 120. The respective one or more other computers 102, 104, 106, 108 may include, for example, one or more server computers or one or more client computers. The client computers may include, for example, a desktop computer, a laptop computer, a smart phone, a tablet, a smart watch, or the like.

Each medical study instrument may include, for example, a form or questionnaire that is provided to a patient in order to solicit information from the patient regarding the patient's experience with a particular medical provider. Each respective medical study instrument may include multiple different sets of questions. For instance, a first set of questions may include questions that are intended to solicit information from patients that may be helpful to other patients or prospective patients when selecting a particular medical facility (e.g., an emergency room, a clinic, hospital, or the like). For example, the first set of questions may include questions such as “In the last 12 months, did you phone this provider's office with a medical question after regular office hours?, “Is this the provider you usually see if you need a check-up, want advise about a health problem, or get sick or hurt?,” “Using any number from 0 to 10, where 0 is the worst provider possible and 10 is the best provider possible, what number would you use to rate this provider?,” “Would you recommend this provider's office to your family and friends?,” or the like.

Alternatively, or in addition, a second set of questions may include questions that are intended to solicit information from patients that may be helpful to a medical provider for evaluating and improving the practice of a medical provider (e.g., a nurse practitioner, a general practitioner, a specialist, a surgeon, or the like). For example, the second set of questions may include questions such as “During your most recent visit, did this provider explain things in a way that was easy to understand?,” “Wait time includes time spent in waiting room and exam room. During your most recent wait, did you see this provider within 15 minutes of your appointment time?,” “Were you seen by a Physician's Assistant or Nurse Practitioner instead of a physician?,” or the like.

The data extraction engine 122 may extract question-answer combinations from each of the plurality of medical study instruments 110, 111, 112, 113, 114, 115, 116, 117. For example, the data extraction engine 122 may extract (i) questions such as those mentioned above and (ii) answers to each respective question from each of the plurality of medical study instruments 110, 111, 112, 113, 114, 115, 116, 117. The extracted question-answer combinations from one or more disparate data sources may be aggregated and stored in the central data storage repository 130.

The data extraction engine 122 may provide data extracted from the obtained data records representing medical study instruments to a classification engine. The classification engine may include one or more software components, one or more hardware components, or a combination thereof (e.g., one or more processors executed programmed logic), that are configured to classify data extracted by the extraction engine 122. For example, the classification engine 122 may classify each extracted question-answer combinations as belonging to one or more particular clusters of question-answer combinations. Clusters of question-answer combinations may include question-answer combinations that are related to aggregated facility data 132, aggregated provider data 134, or the like. For example, the data extraction engine 122 may determine that a first question-answer combination belongs to a facility cluster of questions. In such instances, the data extraction engine 122 may store 122 data related to the question-answer combination in an aggregated facility data table 132. In one implementation, the data extraction engine 120 may determine that the particular question-answer combination should be associated with a particular cluster because, for example, the medical study instrument, the extracted question-answer combination, or both, are tagged with metadata as being related to facility data.

Similarly, for example, the data extraction engine 122 may determine that a second question-answer combination belongs to a provider cluster of questions 134. In such instances, the data extraction engine 122 may store 126 data related to the question-answer combination in an aggregated provider data table 134. In one implementation, the data extraction engine 122 may determine that the particular question-answer combination is tagged with metadata in the corresponding medical study instrument as being related to provider data.

The data extraction engine 122 may extract other types of data from each respective medical study instruments. In some implementations, for example, the data extraction engine 120 can extract data from each respective medical study instrument that can be used to generate a satisfaction score. The satisfaction score may include a facility satisfaction score, a provider satisfaction score, or the like. For example, a facility satisfaction score is numerical value that provides an indication as to a level of satisfaction patients have with respect to a particular medical facility. By way of another example, a provider satisfaction score is a numerical value that provides an indication as to the level of satisfaction patients have with respect to a particular medial provider. The data input by a patient into one or more fields of a medical study instrument may be indicative of the patient's experience with a medical facility, a medical professional, or both, in relation to a particular treatment.

In some implementations, the respective satisfaction scores may be derived from one or more component satisfaction scores such as a quality score, a satisfaction score, a cost score, and/or the like. For example, the provider satisfaction score may be an average of the one or more components scores related to a provider such as a quality score, a satisfaction score, a cost score, or the like. Each respective component score may be based on a predetermined value that corresponds to each of a plurality of patients answers related to the quality of treatment, satisfaction of treatment, and cost of treatment, or the like. Each component score may include, for example, a sum of the predetermined values associated with patient answers. Then, the average of the one or more component scores that may be used as the provider satisfaction score may be an average of the sum for each component score. Alternatively, in some implementations, the satisfaction score may be a weighted average with one or more component scores being assigned a higher (or lower) weight based on the importance (or lack thereof) of the particular component scores in representing the patient's overall health. Regardless of formulation, however, a satisfaction score can be any numerical value that can be used to assign a value to a medical facility or medical provider that can be used to evaluate the medical facility or medical provider, respectively. Once formulated, the data extraction engine 122 may store 124 data related to the respective satisfaction scores in a satisfaction score database such as satisfaction score database 136, e.g., hardware memory.

The centralized data storage repository 130 (e.g., hardware memory) is a data storage unit that provides centralized storage of the aggregated disparate data that has been aggregated, extracted, and classified from each of the multiple medical study instruments 110, 111, 112, 113, 114, 115, 116, 117 obtained by the data extractor unit 122. By way of example, system 100 shows that the aggregated disparate data has been classified into at least two clusters of data that include aggregated facility data table 132 and aggregated provider data table 134. In addition, the centralized data storage repository 130 may also store satisfaction scores for a medical facility, a medical provider, or both, that were respectively derived from data extracted from one or more medical study instruments.

The aggregated facility data table 130 may store data related to a medical facility. The data related to the facility may include data obtained from medical instruments such as medical instruments 110, 111, 112, 113, 114, 115, 116, 117 that is indicative of respective patient's experience at the medical facility. The data related to the facility may include, for example, patient questions 132a, patient answers 132b, patient questionnaire response average 132c, patient questionnaire institutional average 132d, or the like. The data related to the facility may be used to determine facility satisfaction score for the medical facility.

The aggregated provider data table 134 may store data related to a medical provider. The data related to the medical provider may include data obtained from medical instructions such as medical instruments 110, 111, 112, 113, 114, 115, 116, 117 that is indicative of respective patient's experience with the medical provider. The data related to the medical provider may include, for example, patient questions 134a, patient answers 134a, provider average 134c, and institutional average 134d, or the like. The data related to the medical provider may be used to determine a provider satisfaction score for the provider.

The user interface generator 140 may include an executable logic engine that is configured to define a one or more logical relationships 141, 142, 143, 144, 145a, 145b, 146, 147, 148, 149 between one or more respective fields of an aggregated data table and respective regions 162, 164, 166, 172, 174, 176 of a scorecard that can be rendered using the adaptive user interface 150. The executable logic engine may include one or more software components, one or more hardware components, or a combination of both (e.g., one or more processors executing programmed logic) that is configured to define the aforementioned logical relationships 141, 142, 143, 144, 145a, 145b, 146, 147, 148, 149. For example, a scorecard such as a medical facility scorecard 160 that can be rendered using the adaptive user interface 150 may include a plurality of regions such as a questionnaire region 162, a questionnaire statistics region 164, and a satisfaction score region 166. By way of a different example, a scorecard such as a medical provider scorecard 170 that can be rendered using the adaptive user interface 150 may include a plurality of regions such as a questionnaire region 172, a questionnaire statistics region 174, and a satisfaction score region 176.

Each respective scorecard 160, 170 may other types of regions. For example, there may be a comments region that can be juxtaposed with the questionnaire region, the questionnaire statistics region, and the satisfaction score region. For example, the comments region may be rendered underneath the questionnaire region and questionnaire region of each respective scorecard 160, 170. The comments region may include specific comments submitted by respective patients that have sought and received treatment from the entity associated with the scorecard. Yet other types of regions may be employed by the scorecards. For example, there may be a trend region that highlights a trend in entity satisfaction scores by display historical satisfaction scores, a bar graph (or other type of graph) visualizing a trend in score historical satisfaction score movement, or the like. Though each respective scorecard 160, 170 may each include the same region types, the scorecards 160, 170 are distinguishable because each is generated based on a different set of logical mappings between an underlying aggregated data table and respective regions of the scorecard that can be rendered by the adaptive user interface 150.

The user interface generator 140 is configured to process executable program logic that is configured to generate a scorecard such as the scorecard 160, the scorecard 170, the scorecard 300, or the scorecard 400 of FIGS. 1, 3, and 4 respectively using adaptive user 150. The user interface generator 140 may include one or more software components installed on a server computer, a user device, or both, that is configured to communicate with the centralized data storage repository 130 in response to a user for information about an entity such as a facility, a provider, or the like. The user interface generator 140 can select a data set such as an aggregated data table 132 or 134, satisfaction scores database 136, or the like as the source of data that can be displayed in each respective region of an adaptive user interface. Selecting the data set for display may include the user interface generator 140 selecting a particular set of one or more logical mappings from a plurality of available logical mappings 141, 142, 143, 144, 145a, 145b, 145c, 146, 147, 148, 149. Each logical mapping of the plurality of logical mappings 141, 142, 143, 144, 145a, 145b, 145c, 146, 147, 148, 149 may define a logical relationship between particular fields of a user interface such as fields 162a, 162b, 164a, 164b, 164c, 166a and one or more respective fields of an aggregated data table such as aggregated facility data table 132 or 134 such as fields 132a, 132b, 132c, 132d. Alternatively, or in addition, one or more logical mappings such as logical mapping 145a may be established from a field of the satisfaction scores database 136 to a field of an adaptive user interface such as adaptive user interface 160.

The user interface generator 140 can use the executable logic engine to generate rendering data that, when rendered by the user device, causes the user device to render one or more graphical elements that display data stored in the fields of the aggregated data table identified by the set of logical relationships. For example, the user interface generator 140 may include an output port, or other component, that is configured to receive a user request from a client device for a scorecard for an entity. The request may include a key identifier such as an entity name, or other data that identifies the entity. The output port, or other component of the user interface generator 140, may search logical mapping data structures that represent logical mappings of fields of classified data tables stored in the centralized data storage repository 130 to fields of a user interface such as particular fields of a particular region of a scorecard for an entity. Based on the search of the logical mapping data structures using the received key identifier, the output port, or other component of the user interface generator 140, can select a particular data structure representing a particular logical mapping that is associated with the key identifier instead of other logical mapping data structures that represent logical mappings that are not associated with the key identifier. The output port, or other user interface generator 140, may provide the rendering data to a client device in response to a user request for information about an entity such as a facility, a provider, or the like identified by the key identifier that can be processed by the client device to render visual representations of one or more respective regions of a visual scorecard using the set of logical mappings represented by the particular logical mapping data structure.

The output port of the user interface generator may include any combination of software components, hardware components, or a combination of both (e.g., one or more processors executing programmed logic) that are configured to receive and user requests for adaptive user interface displays such as the scorecards disclosed herein, respond to the user request as described above, and then provide rendering data that, when rendered, generates an adaptive user interface such as a scorecard for display on a user device that is responsive to the user request.

By way of example, the adaptive user interface 150 can be used to dynamically configure a display of a user device based on an entity identifier in a request 180 received from a user “Bob” of a user device. For example, in some instances, an entity identifier in a request received from a user device may identify a medical facility. In the example of FIG. 1, the request 180 from “Bob” may include a medical facility identifier “University Hospital.” Bob's user device may transmit the request including the medical facility identifier to the user interface generator 140. The user interface generator 140 may identify an aggregated data table based on the entity identifier included in the request 180, and then identify a set of logical relationships between the identified aggregated data table and the fields of an adaptive user interface. The user interface generator may generate rendering data that can be used to render the adaptive user interface 150 to configure the display of Bob's user device to output 182 a facility scorecard 160. In such instances, the scorecard 160 can be dynamically generated based on rendering data received from the user interface generator 140.

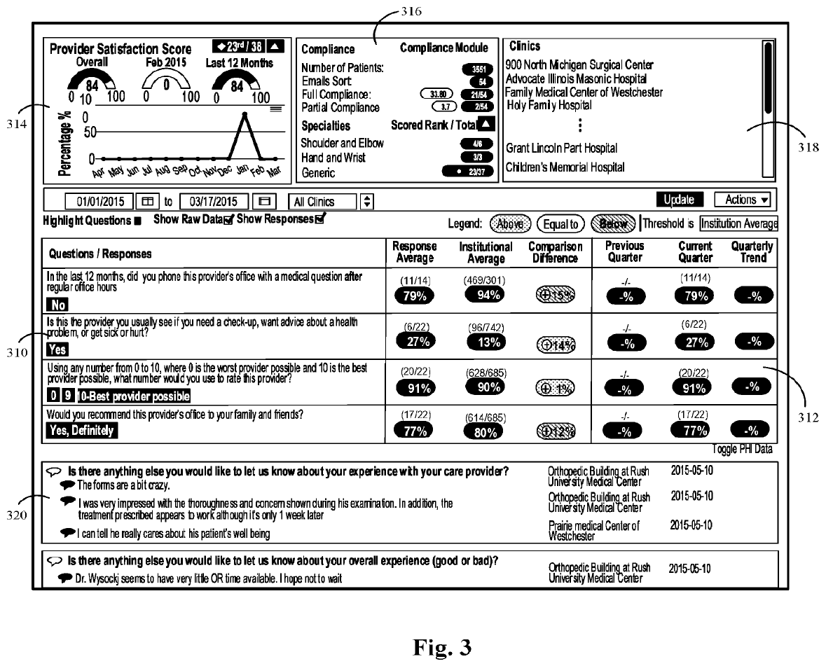

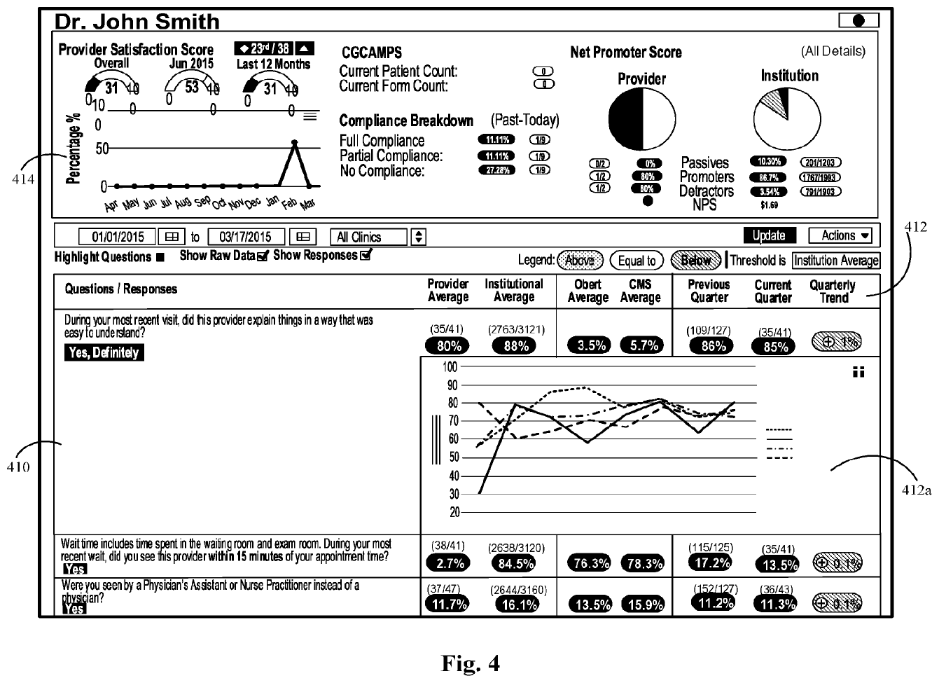

The facility scorecard 160 may display satisfaction data describing historical patient satisfaction data that is associated with an entity identified by the user request that triggered display of the facility scorecard 160. The facility scorecard 160 may be generated based on rendering data received from the user interface generator 140. The rendering data may include data that, when rendered by a user device such as Bob's user device, generates, for display on the user device, a plurality of different user interface regions. The user interface regions may include a questionnaire region 162, a questionnaire statistic region 164, and a satisfaction score region 166. The rendering data generated by the user interface generator 140 can, when rendered, juxtapose the questionnaire region 162, the questionnaire statistic region 164, and the satisfaction score region 166 to generate a satisfaction notification that describes patient satisfaction levels of facility (e.g., University Hospital). In some implementations, rendering data may be configured by the user interface generator 140 to juxtapose, when rendered on the user device, other types of data such as the data and fields shown in FIGS. 3 and 4 with the questionnaire region 162, a questionnaire statistic region 164, and a satisfaction score region 166 in order to generate a satisfaction notification such as facility scorecard 160 in response to a user request.

The questionnaire region 162 may provide, for display, a plurality of questions, and their respective answers, that were asked of patients treated at “University Hospital.” In some implementations, the questionnaire region 162 may display the questions and answers in question and answer pairs from a questionnaire 110, 111, 112, 113, 114, 115, 116, 117. By way of example, a first question may be displayed in the question field 162a and then an answer to the first question from one or more questionnaires 110, 111, 112, 113, 114, 115, 116, 117 may be displayed in the answer field 162b. Though fields for a single question-answer pair are shown in FIG. 1, the questionnaire region 162 may include multiple pairs of fields that can be used to display multiple different question-answer pairs from a questionnaire 110, 111, 112, 113, 114, 115, 116, 117.

The questionnaire statistics region 164 may provide, for display, one or more statistics associated with each question and answer pair displayed in fields 162a, 162b in the questionnaire region 162. In some implementations, the answer provided in answer field 162b may include the most prevalent answer to the question displayed in question field 162a. Alternatively, in other implementations, the questionnaire region 162 may include a first question field 162a that includes a particular question from a questionnaire 110, 111, 112, 113, 114, 115, 116, 117 and a first answer field 162b that includes a particular answer to the particular question from the questionnaire 110, 111, 112, 113, 114, 115, 116, 117 that is displayed in the first question field 162a. In some implementations, the answer in the answer field 162b may be static (e.g., always set to a particular answer of a plurality of answers to the question displayed in question field 162a). Alternatively, the answer in the answer field 162b may be dynamic (e.g., a user may interact with the answer field 162b, change the answer displayed in answer field 162b to a particular answer of the plurality of answers to the potential answers to the question displayed in question field 162a). For dynamic answers, the other scorecard regions such as scorecard regions 164a, 164b, 164c may dynamically update based on the particular answer displayed in the answer field 162b that was input by a user such as Bob who is viewing the scorecard 160.

The questionnaire statistics may include, for example, a response average 164a, an institutional average 164b, and a comparison difference 164c between the response average 164a and the institutional average 164b. The response average 164a may include statistics related to a particular facility. For example, the response average 164a may include the number of questionnaire responders treated at the facility “University Hospital” who submitted an answer (e.g., any answer) to the question in question field 162a. Alternatively, the response average 164a may include, for example, a number of questionnaire responders treated at the facility “University Hospital” who submitted a particular answer (e.g., yes, no, “5”, or the like) to the question displayed in question field 162a. For example, if a user selects the answer “no” in answer field 162b for a particular question in question field 162a, then the response average 162a can be updated to indicate the number of responders from the facility “University Hospital” who selected the answer “no” for the particular question. The response average may include a proportion such as the number of responders that were treated at the facility “University Hospital” who answered the question divided by the total number of patients treated at the facility “University Hospital,” a number of responders treated at the facility “University Hospital” who provided a particular answer divided by the number of persons who were treated at the facility “University Hospital” and responded to the questionnaire that includes the particular question, or the like.

The institutional average 164b may similarly include a number that is representative of the number of patients who responded to a questionnaire that included the question, the number of patients who provided a particular answer to the question of the questionnaire, or the like. However, institutional average may be determined by determination a proportion that is measured with respect to the number of questionnaire responders who submitted an answer in an answer field 162b to the question in a question field 162a from a number of other facilities that are similar in offerings, size, and scope to “University Hospital.” Such a proportion may include, for example, a number of responders from other facilities that are similar in offerings, size, and scope to “University Hospital” divided by the total number of patients treated at facilities that are similar in offerings, size, and scope to “University Hospital.” Alternatively, such a proportion may include, for example, a number of responders from other facilities that are similar in offerings, size, and scope to “University Hospital” that provided a particular answer in an answer field 162b to a particular question 162a divided by the total number of responders from other facilities that are similar in offerings, size, and scope to “University Hospital” that responded to the questionnaire that included the particular question. Other types of proportions can also be used to represent the institutional average 164b.

The comparison difference 164c can be used to display the percentage difference between the response average 164a and the institutional average 164b. In some implementations, the portion of the questionnaire statistics region 164 that is associated the comparison difference 164c can visually altered if the difference between the response average 164a and the institutional average 164b satisfies a predetermined threshold. Visually altering may include, for example, changing the color of the text in the region 164, changing the script of the text in the region 164, changing the background color of the region 164, or any other form of visual alteration of region 164, the displayed data in region 164, or the like.

By way of example, the adaptive user interface 150 can be used to dynamically configure a display of a user device based on an entity identifier in a request 190 received from a user “Tom” of a user device. For example, in some instances, an entity identifier in a request received from a user device may identify a provider identifier. In the example of FIG. 1, the request 190 from “Tom” may include a provider identifier “Dr. Smith.” Tom's user device may transmit the request including the provider identifier to the user interface generator 140. The user interface generator 140 may identify an aggregated data table based on the entity identifier included in the request 190, and then identify a set of logical relationships between the identified aggregated data table and the fields of an adaptive user interface. The user interface generator may generate rendering data that can be used to render the adaptive user interface 150 to configure the display of Tom's user device to output 192 a provider scorecard 170. In such instances, the scorecard 170 can be dynamically generated based on rendering data received from the user interface generator 140.

The provider scorecard 170 may display satisfaction data describing historical patient satisfaction data that is associated with an entity identified by the user request that triggered display of the provider scorecard 170. The provider scorecard 170 may be generated based on rendering data received from the user interface generator 140. The rendering data may include data that, when rendered by a user device such as Tom's user device, generates, for display on the user device, a plurality of different user interface regions. The user interface regions may include a questionnaire region 172, a questionnaire statistic region 174, and a satisfaction score region 176. The rendering data generated by the user interface generator 140 can, when rendered, juxtapose the questionnaire region 172, the questionnaire statistic region 174, and the satisfaction score region 176 to generate a satisfaction notification that describes patient satisfaction levels associated with the provider (e.g., Dr. Smith). In some implementations, rendering data may be configured by the user interface generator 140 to juxtapose, when rendered on the user device, any of the additional data regions and fields shown in FIGS. 3 and 4 with the questionnaire region 172, a questionnaire statistic region 174, and a satisfaction score region 176 in order to generate a satisfaction notification such as provider scorecard 170 in response to a user request.

The questionnaire region 172 may provide, for display, a plurality of questions, and their respective answers, that were asked of patients treated by a particular provider “Dr. Smith.” In some implementations, the questionnaire region 172 may display the questions and answers in question and answer pairs from a questionnaire 110, 111, 112, 113, 114, 115, 116, 117. By way of example, a first question may be displayed in the question field 172a and then an answer to the first question from one or more questionnaires 110, 111, 112, 113, 114, 115, 116, 117 may be displayed in the answer field 172b. Though fields for a single question-answer pair are shown in FIG. 1, the questionnaire region 172 may include multiple pairs of fields that can be used to display multiple different question-answer pairs from a questionnaire 110, 111, 112, 113, 114, 115, 116, 117.

The questionnaire statistics region 174 may provide, for display, one or more statistics associated with each question and answer pair displayed in fields 172a, 172b in the questionnaire region 172. In some implementations, the answer provided in answer field 172b may include the most prevalent answer to the question displayed in question field 172a. Alternatively, in other implementations, the questionnaire region 172 may include a first question field 172a that includes a particular question from a questionnaire 110, 111, 112, 113, 114, 115, 116, 117 and a first answer field 172b that includes a particular answer to the particular question from the questionnaire 110, 111, 112, 113, 114, 115, 116, 117 that is displayed in the first question field 172a. In some implementations, the answer in the answer field 172b may be static (e.g., always set to a particular answer of a plurality of answers to the question displayed in question field 172a). Alternatively, the answer in the answer field 172b may be dynamic (e.g., a user may interact with the answer field 172b, change the answer displayed in answer field 172b to a particular answer of the plurality of answers to the potential answers to the question displayed in question field 172a). For dynamic answers, the other scorecard regions such as scorecard regions 174a, 174b, 174c may dynamically update based on the particular answer displayed in the answer field 172b that was input by a user such as Bob who is viewing the scorecard 170.

The questionnaire statistics may include, for example, a response average 174a, an institutional average 174b, and a comparison difference 174c between the response average 174a and the institutional average 174b. The response average 174a may include statistics related to a particular provider. For example, the response average 174a may include the number of questionnaire responders treated by the provider “Dr. Smith” who submitted an answer (e.g., any answer) to the question in question field 172a. Alternatively, the response average 174a may include, for example, a number of questionnaire responders treated by the provide “Dr. Smith” who submitted a particular answer (e.g., yes, no, “5”, or the like) to the question displayed in question field 172a. For example, if a user selects the answer “no” in answer field 172b for a particular question in question field 172a, then the response average 172a can be updated to indicate the number of responders treated by “Dr. Smith” who selected the answer “no” for the particular question. The response average may include a proportion such as the number of responders that were treated by the provider “Dr. Smith” who answered the question divided by the total number of patients treated by “Dr. Smith,” a number of responders treated “Dr. Smith” who provided a particular answer to a particular question divided by the number of persons who were treated by “Dr. Smith” and responded to the questionnaire that includes the particular question, or the like.

The institutional average 174b may similarly include a number that is representative of the number of patients who responded to a questionnaire that included the question, the number of patients who provided a particular answer to the question of the questionnaire, or the like. However, institutional average 174b may be determined using a proportion that is measured with respect to the number of questionnaire responders who submitted an answer in an answer field 172b to the question in a question field 172a were treated by a provider that practices in substantially the same field, substantially the same practice, or the like as “Dr. Smith.” Such a proportion may include, for example, a number of responders treated by a provider that practices in substantially the same field, substantially the same practice, or the like as “Dr. Smith.” divided by the total number of patients treated by a provider that practices in substantially the same field, substantially the same practice, or the like as “Dr. Smith.” Alternatively, such a proportion may include, for example, a number of responders treated by a provider that practices in substantially the same field, substantially the same practice, or the like as “Dr. Smith.” that provided a particular answer in an answer field 172b to a particular question 172a divided by the total number of responders treated by a provider that practices in substantially the same field, substantially the same practice, or the like as “Dr. Smith” that responded to the questionnaire that included the particular question. Other types of proportions can also be used to represent the institutional average 174b.

The comparison difference 174c can be used to display the percentage difference between the response average 174a and the institutional average 174b. In some implementations, the portion of the questionnaire statistics region 174 that is associated with the comparison difference 174c can be visually altered if the difference between the response average 174a and the institutional average 174b satisfies a predetermined threshold. Visually altering may include, for example, changing the color of the text in the region 174, changing the script of the text in the region 174, changing the background color of the region 174, or any other form of visual alteration of region 174, the displayed data in region 174, or the like.

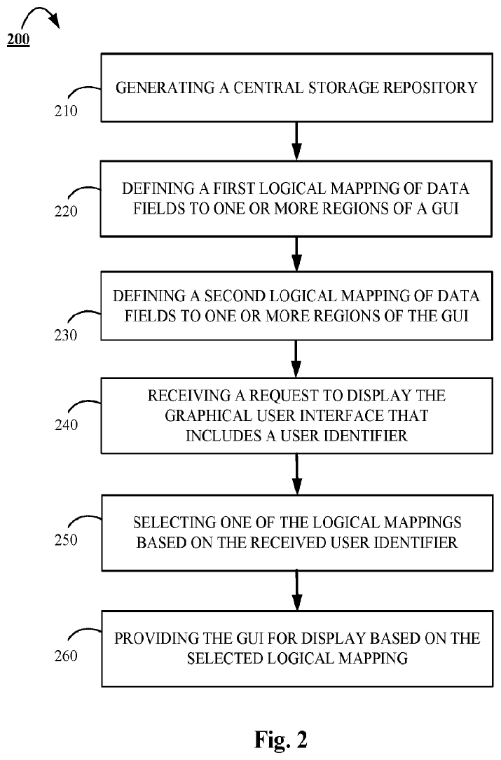

FIG. 2 is a flowchart of an example of a process 200 for implementing adaptive user interfaces. Generally, the process 200 may include generating 210 a central storage repository, defining 220 a first logical mapping of data fields to one or more regions of a graphical user interface (GUI), defining a 230 a second logical mapping of data fields to one or more regions of the graphical user interface (GUI), receiving 240 a request to display the graphical user interface (GUI), wherein the user interface includes a user identifier, selecting 250 one of the logical mappings based on the received user identifier, and providing 260 the graphical user interface (GUI) for display based on the selected logical mapping. For convenience, the process 200 will be described as being performed by a system such as the system 100 of FIG. 1.

The system may begin performance of the process 200 by generating 210 a central data storage repository that aggregates data that has been extracted from obtained data records representing multiple different medical study instruments, wherein the aggregated data is classified into a first type of data and a second type of data. The first type of data may include facility satisfaction data. Facility satisfaction data may include data that has been obtained from one or more medical instruments related to a patient's satisfaction with a particular facility (e.g., a particular hospital, clinic, or the like). The second type of data may include provider satisfaction data. Provider satisfaction data may include data has been obtained from one or more medical study instruments related to a patient's experience with a particular provider (e.g., a particular doctor, practitioner, or the like).

In some implementations, the system may generate a central storage repository that aggregates data that has been extracted from multiple different medical study instruments by obtaining a plurality of populated medical study instruments from a plurality of different computer and extracting one or more question-answer pairs from the obtained medical study instruments. The system may determine a classification of each extracted question-answer pairs. Determining a classification of each extracted question-answer pair may include, for example, determining whether the extracted question-answer pair is related to a facility, a provider, or both. The system can save the question-answer pair into an aggregated data storage area based on the determined classification.

The system can define 220 one or more first logical mappings from each of a plurality of fields in the first type of data to one or more respective regions of a graphical user interface. For example, the system can define a logical mapping from each field of a first aggregated data table to respective fields of respective regions of an adaptive user interface that can be used to configure a user interface of user device to display a scorecard. In some implementations, the first aggregated data table may include a facility aggregated data table storing facility satisfaction data that has been extracted from one or more medical study instruments.

The system can define 230 one or more second logical mappings from each of a plurality of fields in the second type of data to one or more respective regions of the graphical user interface. For example, the system can define a logical mapping from each field of a second aggregated data table to respective fields of respective regions of an adaptive user interface that can be used to configure a user interface of a user device to display a scorecard. In some implementations, the second aggregated data table may include a provider aggregated data table storing facility satisfaction data that has been extracted from one or more medical study instruments.

The system can receive 240 a request to display the graphical user interface from a user of a user device, wherein the request includes an entity identifier. For example, the system can receive a request to display a satisfaction scorecard for a facility. The request may include, for example, an identifier of a facility such as a hospital name, a clinic name, or the like. Alternatively, the system can receive a request to display a satisfaction scorecard for a provider. In such instances, the request may include an identifier of a medical provider such as a doctor, a nurse practitioner, a nurse, a therapist, or the like.

The system can select 250 either the first logical mapping or the second logical mapping based on the received entity identifier. In some implementations, for example, the first logical mapping may associate a first aggregated data table with fields of a user interface and a second logical mapping may associate a second aggregated data table with fields of a user interface. By way of a specific example, the first logical mapping may associate a facility satisfaction aggregated data table with a region of fields in a user interface and the second logical mapping may associate a provider satisfaction aggregated data table with a region of fields in a user interface. In such instances, the system can select the logical mapping corresponding to the facility satisfaction aggregated data table if the received entity identifier identifies a facility. Alternatively, the system can select the logical mapping corresponding to the provider aggregated data table if the received entity identifier identifies a provider.

The system can provide 260 the graphical user interface for display, wherein the one or more respective regions are populated with data based on the selected logical mapping. Providing the graphical user interface for display may include, generating rendering data that can be used to render the graphical user interface based on the selected logical mappings. The generated rendering data juxtapose respective regions of user interface as described with reference to scorecards of FIGS. 1, 3, and 4. The generated rendering data may be transmitted to the user device, which can render the rendering data to generate a scorecard having regions organized as described with reference to FIGS. 1, 3, and 4.

FIG. 3 is an example of a scorecard 300 that is generated for a medical facility. The scorecard 300 is an example of a scorecard that can be rendered for display on a user device using the disclosed adaptive user interface. The scorecard 300 may include a questionnaire region 310, a questionnaire statistics region 312, a facility (or provider) satisfaction score region 314, a compliance region 316, a facility (or provider) input region 318, and a comments region 320.

FIG. 4 is an example of a scorecard 400 that may be generated for a medical provider. The scorecard 400 is an example of a scorecard that can be rendered for display on a user device using the disclosed adaptive user interface. The scorecard 400 may include a questionnaire region 410, a questionnaire statistics region 412, and a provider (or facility) satisfaction score region 414.

Embodiments of the subject matter, the functional operations and the processes described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps may be provided, or steps may be eliminated, from the described processes. Accordingly, other implementations are within the scope of the following claims.

What is claimed is:

1. A data processing system for processing data records and searching data structures at least partly generated from the processed data records, comprising:
an input port that retrieves, from multiple, different data stores, data records, with each data record representing a medical study instrument;
an extraction engine that processes the data records by extracting specified portions of data included in each of the data records retrieved over the input port;
a central data storage repository that aggregates the extracted data that has been extracted from the data records representing respective multiple different medical study instruments;
a classification engine that classifies a first portion of the aggregated data into a first type of data and a second portion of the aggregated data into a second type of data;
an executable logic engine that defines a first logical mapping from each of a plurality of fields in the first type of data to one or more respective regions of a graphical user interface and that further defines a second logical mapping from each of a plurality of fields in the second type of data to one or more respective regions of the graphical user interface; and
an output port that:
receives a request to display the graphical user interface from a user of a user device, wherein the request includes a key identifier;
searches data structures representing the first and second logical mapping, wherein at least one of the searched data records includes a key identifier;
responsive to searching, selects either the first logical mapping or the second logical mapping based on the received key identifier; and
providing data for the graphical user interface that when rendered on a display device renders one or more visual representations of the one or more respective regions populated with data based on the selected logical mapping.

2. The data processing system of claim 1, wherein at least one first region of the graphical user interface is populated with one or more question-answer combinations based on the selected logical mapping.

3. The data processing system of claim 2, wherein at least one second region of the graphical user interface is populated with data that is indicative of the number of people who have provided the same answer to the same question set forth in the particular question-answer combination.

4. The data processing system of claim 3, wherein the second region of the graphical user interface further comprises:
a first displayed value that is indicative of the number of people at a particular medical facility that provided the same answer to the same question set forth in the particular question-answer combination;
a second displayed value that is indicative of the number of people at multiple different medical facilities that provided the same answer to the same questions set forth in the particular question-answer combination; and
a third displayed value that is indicative of the difference between the first displayed value and the second displayed value.

5. The system of claim 4, wherein the output port is further configured to:
determine whether the third displayed value exceeds a predetermined threshold; and
in response to a determination that the third displayed value exceeds a predetermined threshold, highlight a predetermined proximity of the graphical user interface associated with the third displayed value using a first color.

6. The system of claim 4, wherein the output port is further configured to:
determine whether the third displayed value falls below a predetermined threshold; and
in response to a determination that the third displayed value falls below a predetermined threshold, highlighting a predetermined proximity of the graphical user interface associated with the third displayed value using a second color that is different than the first color.

7. A system comprising:
one or more data processing apparatus; and
one or more computer-readable storage devices having stored thereon instructions that, when executed by the one or more data processing apparatus, cause the one or more data processing apparatus to perform operations comprising:
generating a central data storage repository that aggregates data that has been extracted from multiple different medical study instruments, wherein the aggregated data is classified into a first type of data and a second type of data;
defining a first logical mapping from each of a plurality of fields in the first type of data to one or more respective regions of a graphical user interface;
defining a second logical mapping from each of a plurality of fields in the second type of data to one or more respective regions of the graphical user interface;
receiving a request to display the graphical user interface from a user of a user device, wherein the request includes an entity identifier;
selecting either the first logical mapping or the second logical mapping based on the received entity identifier; and
providing the graphical user interface for display, wherein the one or more respective regions are populated with data based on the selected logical mapping.

8. The system of claim 7, wherein generating a central repository that aggregates data that has been extracted from multiple different medical study instruments further comprises:
obtaining a plurality of populated medical study instruments from a plurality of different computers;
extracting one or more question-answer pairs from the obtained medical study instruments;
determining a classification of each extracted question-answer pairs; and
saving the question-answer pair into an aggregated data storage area based on the determined classification.

9. The system of claim 7, wherein at least one first region of the graphical user interface is populated with one or more question-answer combinations based on the selected logical mapping.

10. The system of claim 9, wherein at least one second region of the graphical user interface is populated with data that is indicative of the number of people who have provided the same answer to the same question set forth in the particular question-answer combination.

11. The system of claim 10, wherein the second region of the graphical user interface further comprises:
a first displayed value that is indicative of the number of people at a particular medical facility that provided the same answer to the same question set forth in the particular question-answer combination;
a second displayed value that is indicative of the number of people at multiple different medical facilities that provided the same answer to the same questions set forth in the particular question-answer combination; and
a third displayed value that is indicative of the difference between the first displayed value and the second displayed value.

12. The system of claim 11, further comprising:
determining whether the third displayed value exceeds a predetermined threshold; and
in response to determining that the third displayed value exceeds a predetermined threshold, highlighting a predetermined proximity of the graphical user interface associated with the third displayed value using a first color.

13. The system of claim 11, further comprising:
determining whether the third displayed value falls below a predetermined threshold; and
in response to determining that the third displayed value falls below a predetermined threshold, highlighting a predetermined proximity of the graphical user interface associated with the third displayed value using a second color that is different than the first color.

14. A method comprising:
generating a central data storage repository that aggregates data that has been extracted from multiple different medical study instruments, wherein the aggregated data is classified into a first type of data and a second type of data;
defining a first logical mapping from each of a plurality of fields in the first type of data to one or more respective regions of a graphical user interface;
defining a second logical mapping from each of a plurality of fields in the second type of data to one or more respective regions of the graphical user interface;
receiving a request to display the graphical user interface from a user of a user device, wherein the request includes an entity identifier;
selecting either the first logical mapping or the second logical mapping based on the received entity identifier; and
providing the graphical user interface for display, wherein the one or more respective regions are populated with data based on the selected logical mapping.

15. The method of claim 14, wherein generating a central repository that aggregates data that has been extracted from multiple different medical study instruments, further comprises:
obtaining a plurality of populated medical study instruments from a plurality of different computers;
extracting one or more question-answer pairs from the obtained medical study instruments;
determining a classification of each extracted question-answer pairs; and
saving the question-answer pair into an aggregated data storage area based on the determined classification.

16. The method of claim 14, wherein at least one first region of the graphical user interface is populated with one or more question-answer combinations based on the selected logical mapping.

17. The method of claim 16, wherein at least one second region of the graphical user interface is populated with data that is indicative of the number of people who have provided the same answer to the same question set forth in the particular question-answer combination.

18. The method of claim 17, wherein the second region of the graphical user interface further comprises:
a first displayed value that is indicative of the number of people at a particular medical facility that provided the same answer to the same question set forth in the particular question-answer combination;
a second displayed value that is indicative of the number of people at multiple different medical facilities that provided the same answer to the same questions set forth in the particular question-answer combination; and
a third displayed value that is indicative of the difference between the first displayed value and the second displayed value.

19. The method of claim 18, further comprising:
determining whether the third displayed value exceeds a predetermined threshold; and
in response to determining that the third displayed value exceeds a predetermined threshold, highlighting a predetermined proximity of the graphical user interface associated with the third displayed value using a first color.

20. The method of claim 18, further comprising:
determining whether the third displayed value falls below a predetermined threshold; and
in response to determining that the third displayed value falls below a predetermined threshold, highlighting a predetermined proximity of the graphical user interface associated with the third displayed value using a second color that is different than the first color.

21. A non-transitory computer-readable storage device having stored thereon instructions, which, when executed by a data processing apparatus, cause the data processing apparatus to perform operations comprising:
generating a central data storage repository that aggregates data that has been extracted from multiple different medical study instruments, wherein the aggregated data is classified into a first type of data and a second type of data;
defining a first logical mapping from each of a plurality of fields in the first type of data to one or more respective regions of a graphical user interface;
defining a second logical mapping from each of a plurality of fields in the second type of data to one or more respective regions of the graphical user interface;
receiving a request to display the graphical user interface from a user of a user device, wherein the request includes an entity identifier;
selecting either the first logical mapping or the second logical mapping based on the received entity identifier; and
providing the graphical user interface for display, wherein the one or more respective regions are populated with data based on the selected logical mapping.

22. The non-transitory computer-readable storage device of claim 21, wherein generating a central repository that aggregates data that has been extracted from multiple different medical study instruments, further comprises:
obtaining a plurality of populated medical study instruments from a plurality of different computers;
extracting one or more question-answer pairs from the obtained medical study instruments;
determining a classification of each extracted question-answer pairs; and
saving the question-answer pair into an aggregated data storage area based on the determined classification.

23. The non-transitory computer-readable storage device of claim 21, wherein at least one first region of the graphical user interface is populated with one or more question-answer combinations based on the selected logical mapping.

24. The non-transitory computer-readable storage device of claim 23, wherein at least one second region of the graphical user interface is populated with data that is indicative of the number of people who have provided the same answer to the same question set forth in the particular question-answer combination.

25. The non-transitory computer-readable storage device of claim 24, wherein the second region of the graphical user interface further comprises:
a first displayed value that is indicative of the number of people at a particular medical facility that provided the same answer to the same question set forth in the particular question-answer combination;
a second displayed value that is indicative of the number of people at multiple different medical facilities that provided the same answer to the same questions set forth in the particular question-answer combination; and
a third displayed value that is indicative of the difference between the first displayed value and the second displayed value.

26. The non-transitory computer-readable medium of claim 25, the operations further comprising:
determining whether the third displayed value exceeds a predetermined threshold; and
in response to determining that the third displayed value exceeds a predetermined threshold, highlighting a predetermined proximity of the graphical user interface associated with the third displayed value using a first color.

27. The non-transitory computer-readable medium of claim 25, the operations further comprising
determining whether the third displayed value falls below a predetermined threshold; and
in response to determining that the third displayed value falls below a predetermined threshold, highlighting a predetermined proximity of the graphical user interface associated with the third displayed value using a second color that is different than the first color.
